(19) United States
(12) Patent Application Publication
(10) Pub. No.: US 2009/0068276 A1
(43) Pub. Date: Mar. 12, 2009

(54) Pharmaceutical compositions

(75) Inventors: Mark Jonathan Main, Chippenham (GB); Martin James Oliver, Chippenham (GB); Timothy Wright, Chippenham (GB)

Correspondence Address: Davidson, Davidson & Kappel, LLC, 485 7th Avenue, 14th Floor, New York, NY, 10018, US

(73) Assignee: Vectura Group plc, Chippenham (GB)

(21) Appl. No.: 12/154,415

(22) Filed: May 22, 2008

(30) Foreign Application Priority Data
May 22, 2007 (GB) 0709811.4

(51) Int. Cl. A61K 9/14; A61K 31/55; A61P 11/00

(52) U.S. Cl. 424/490; 514/217; 424/489

(57) ABSTRACT

The present invention relates to new pharmaceutical formulations comprising pharmaceutically active agents which can induce one or more involuntary coughs in a patient when administered as conventional formulations and/or via conventional routes. Formulations are provided comprising a cough-inducing pharmaceutically active agent, wherein the formulation may be administered by pulmonary inhalation without inducing a cough. Alternatively, formulations are provided for administering the cough-inducing active agent via an alternative route.

The present invention relates to new pharmaceutical formulations comprising pharmaceutically active agents which can induce one or more involuntary coughs in a patient when administered as conventional formulations and/or via conventional routes.

By cough it is meant a sudden, often repetitive, spasmodic contraction of the thoracic cavity, resulting in sudden expulsion of air from the lungs, and usually accompanied by a distinctive sound.

Anatomical and environmental factors mean that the airways and lungs are under constant threat of exposure to a variety of harmful airborne substances, as well as aspirated gastric contents or accidental inhalation of foodstuffs. A variety of defensive mechanisms have evolved along with the normal function of the respiratory system to help protect against such exposure. This airway protection relies upon specialized epithelial barriers and immune responses, as well as a variety of highly coordinated neural reflex responses that help to limit the degree of potential harm and hopefully expel the harmful substance from the airways. Perhaps the most widely recognized neural response involved in airway protection is coughing.

Airway sensory nerves (sometimes called cough receptors) are located near the surface of the upper and lower respiratory tract. Various agents, including noxious gases and fumes, foreign bodies and other irritants can stimulate these nerves to send signals to the brain, which in turn triggers a response involving a deep inhalation and then a forced exhalation, i.e. a cough. Chemicals produced in the body, such as substance P and bradykinin, can stimulate the cough reflex.

Airway sensory nerves can be broadly classified in accordance with their function and they generally fall into two categories, those that are primarily mechanically sensitive (low threshold mechanosensors) and those that are primarily chemically sensitive (chemosensors or alternatively, nociceptors). Low threshold mechanoreceptors are readily activated by one or more mechanical stimuli, including lung inflation, bronchospasm or light touch, but generally do not respond directly to chemical stimuli. On the other hand, chemosensors are typically activated directly or sensitized by a wide range of chemicals, including capsaicin, bradykinin, adenosine, PGE2, but are relatively insensitive to mechanical stimuli.

A variety of medical conditions are possible causes of chronic cough namely, smoker's cough, asthma, chronic bronchitis, emphysema, COPD, lung cancer, stress, GERD, habitual cough, enlarged uvula, common cold, post-infection cough, upper respiratory infection, viral respiratory infection, bacterial respiratory infection, sinusitis, postnasal drip, upper airway obstruction, tracheoesophageal fistula (cough when eating), pneumonia, Pneumococcal pneumonia, heart failure, foreign object in lung, pulmonary edema, congestive heart failure, tuberculosis, bronchiectasis, pulmonary embolism, pulmonary fibrosis and lung abscess.

It has long been recognized that the administration of some pharmaceutically active agents elicits an involuntary cough. For example, it has been observed that the inhalation of formulations comprising fentanyl or capsaicin can cause this unwanted side effect. In some situations, the patient experiences just a single cough following inhalation of the pharmaceutical formulation. Occasionally, a coughing episode may be triggered, lasting a significant period of time and causing the patient serious discomfort and distress. Whilst this does not mean that the formulation is not effective in eliciting the desired therapeutic effect, the unwanted involuntary cough can mean that the formulation is less attractive as a commercial product.

Cough inducing medications or substances known to cause chronic cough include Accupril, Accuretic 10/12.5, Accuretic 20/12.5, ACE inhibitors, Acenorm, Aceon, Alphapril, Altace, Amprace, Apo-Enalapril, Asig, Auspril, Benazepril, Benazepril Hydrochloride, Captohexal, Captopril (Capoten), Citrate, Chloride, Coversyl, Coversyl Plus, Enahexal, Enaladil, Enalapril, Enalapril (Vasotec), Enzace, Fibsol, Fosinopril, Giloten, Gopten, Lexxel, Liprace, Lisinopril, Lisinopril (Zestril, Prinivil), Lisodur, Lotensin, Mavik, Moexipril, Monoplus, Monopril, Odrik, Prinivil, Prinvil, Quinapril, Ramace, Renitec, Renitec Plus, Tarka, Trandolapril, Tritace, Univasc, Vasotec and Zestril.

Paradoxical bronchospasm is a phenomenon experienced when a prescribed bronchospasm therapy actually causes bronchospasm. Bronchospasm is a sudden constriction of the muscles in the walls of the bronchioles resulting in difficulty in breathing. The hypersensitivity of the muscles in the bronchiole walls is a result of exposure to a stimulus which under normal circumstances would cause little or no response. The resulting constriction and inflammation causes a narrowing of the airways and an increase in mucous production. Consequently the amount of oxygen available to the individual decreases thereby causing breathlessness, coughing and hypoxia.

The present invention is concerned with the provision of compositions comprising formulation strategies that avoid side effects such as cough, whilst maintaining therapeutic efficacy.

The inventors have now discovered that clomipramine (Anafranil®) also induces an involuntary cough when it is administered via pulmonary inhalation. Clomipramine is a member of the class of drugs generally referred to as tricyclic antidepressants, which are generally prescribed for the treatment of severe depression or depression which occurs with anxiety. Clomipramine has also been suggested for the treatment of premature ejaculation.

Although clomipramine is commercially available in the form of oral tablets or capsules, the onset of its therapeutic effect is relatively slow when it is administered via the oral route. In contrast, a much faster onset of its effect may be observed when it is administered by pulmonary inhalation, and this rapid onset of its effect is especially attractive when clomipramine is used to treat premature ejaculation.

It would appear that the unwanted cough associated with the administration of certain pharmaceutically active agents such as clomipramine is due to stimulation of chemosensors, rather than mechanosensors. The cough is drug-specific and otherwise identical formulations administered in the same manner do not elicit the cough response.

It is therefore an aim of the present invention to provide a formulation comprising a cough-inducing pharmaceutically active agent that will not elicit an involuntary cough in the patient. The formulation may be administered by inhalation or an alternative route, which is preferably one which will achieve a rapid onset of the therapeutic effect.

The present invention concerns a formulation comprising a cough-inducing pharmaceutically active agent, wherein the formulation may be administered by pulmonary inhalation without inducing a cough.

In the present application, a cough-inducing pharmaceutically active agent is an agent which may induce a cough upon administration, for example upon administration by pulmonary inhalation. The cough is preferably induced by a chemical trigger (stimulation of a chemosensor) and not a mechanical trigger (stimulation of a mechanosensor).

In an embodiment, the cough-inducing pharmaceutically active agent is clomipramine.

It is speculated that the chemosensors that are responsible for drug-induced coughs may only be found in certain parts of the respiratory tract and that bronchial tubes in the smaller branches and the alveoli do not have such sensors or receptors. Therefore, in a first aspect of the present invention, the formulation comprises the pharmaceutically active agent in a form that will target deposition in certain parts of the respiratory tract and can therefore minimize or avoid stimulation of the chemosensors which trigger the unwanted involuntary cough.

The formulation according to the first aspect of the present invention comprises the pharmaceutically active agent in the form of particles having a size which will allow and encourage deposition in the alveoli whilst avoiding and reducing deposition in parts of the respiratory tract where the chemosensors are thought to be located, such as the upper airway. To that end, preferably, the D₅₀ of the formulation comprising the pharmaceutically active agent is less than 2 μm, preferably less than 1.5 μm and most preferably less than 1 μm. It is believed that the pattern of deposition of active particles contained in a formulation with a D₅₀ of approximately 2 μm following inhalation is such that particles of this size will still induce an involuntary cough when inhaled.

D₅₀ represents the median, or the 50^{th} percentile, of the particle size distribution, based upon the particles' volume equivalent diameter. Thus, a D₅₀ of 1 μm means that 50% of the particles in a formulation have a volume equivalent diameter of 1 μm or less (‘equivalent’ diameter assumes the particles are spherical for convenient comparison). The D₅₀ value of a formulation may be obtained by measuring the volume equivalent diameter of the particles in the formulation using particle sizing equipment (for example, Malvern Mastersizer 2000) and plotting the data on a cumulative particle size distribution graph (percentage versus volume equivalent diameter). The D₅₀ value can be read from the graph where the 50% horizontal line intersects the particle size distribution curve.

When dry powders are produced using conventional processes, the active particles will vary in size, and often this variation can be considerable. This can make it difficult to ensure that a high enough proportion of the active particles are of the appropriate size to ensure deposition at a particular site within the respiratory tract upon administration by pulmonary inhalation. In certain circumstances, such as circumstances where reproducible and accurate deposition of an inhaled formulation in the lung is desired, it may therefore be advantageous to have a dry powder formulation wherein the size distribution of the active particles is narrow. For example, the geometric standard deviation of the active particle aerodynamic or volumetric size distribution (σg) may preferably be not more than 2, more preferably not more than 1.8, not more than 1.6, not more than 1.5, not more than 1.4 or even not more than 1.2. A high kurtosis value ensures that doses are both reproducible with respect to the active agent content and that the dose is delivered to the same region of the lung on each delivery ensuring a reproducible pharmacokinetic profile. One property related to the particle size distribution of a collection of particles is the kurtosis and the approach of using Kurtosis to describe a particle size distribution is well established in the pharmaceutical sciences (see, for example, Staniforth J. N. (1988), Pharmaceutics The Science of Dosage Form Design, Ed. Aulton, M. E., Churchill Livingstone, ISBN:0443036438. The symmetry of a distribution is based on a comparison of the height or thickness of the tails of the distribution curve and the “sharpness” of the peaks with those of a normal distribution. “Thick” tailed, “sharp” peaked curves are described as leptokurtic whereas “thin” tailed, “blunt” peaked curves are platykurtic and the normal distribution is mesokurtic. The normalised coefficient of kurtosis has a value of 0 for a mesokurtic normal distribution, a negative value for curves showing platykurtosis and positive values for leptokurtic size distributions. This may improve dose efficiency and reproducibility.

Formulations according to the first aspect of the present invention may be produced in a suitable particle size (i.e. having a D₅₀ value of preferably less than 2 μm, more preferably less than 1.5 μm and most preferably less than 1 μm) using high intensity milling techniques such as jet milling or Mechano-Fusion (also known as mechano-chemical bonding). The production of dry powder formulations comprising clomipramine and having a D₅₀ of approximately 1 μm is described in the Examples. Inhalation of clomipramine formulations such as those described in the Examples is not believed to induce an involuntary cough because the small clomipramine particles deposit deep in the airway where chemosensors responsible for drug-induced coughs are not thought to be found.

In a second aspect of the invention, the active particles may also or alternatively be protected to reduce the likelihood of their stimulating the chemosensors upon inhalation. Such protection could, for example, take the form of a coating which will preferably be removed, for example by dissolution, upon deposition of the particle in the lung, but which will provide at least an initial (albeit perhaps short-lived) barrier between the pharmaceutically active agent and the chemosensor.

One example of a suitable coating is a layer of an inert material that will provide a protective layer. Suitable materials for this purpose include the additives and force control agents discussed in earlier patent applications such as those published as WO 96/23485, WO 97/03649 and WO 2004/093848. Some of the preferred coating materials are discussed below.

The coating material may comprise or consist of a metal stearate, for example, zinc stearate, magnesium stearate, calcium stearate, sodium stearate or lithium stearate, or a derivative thereof, for example, sodium stearyl fumarate or sodium stearyl lactylate. Magnesium stearate is a preferred coating material.

The coating material may comprise or consist of one or more surface active materials, in particular materials that are surface active in the solid state, which may be water soluble or water dispersible, for example lecithin, in particular soya lecithin, or substantially water insoluble, for example solid state fatty acids such as oleic acid, lauric acid, palmitic acid, stearic acid, erucic acid, behenic acid or derivatives (such as esters and salts) thereof, such as glyceryl behenate. Specific examples of such surface active materials are phosphatidylcholines, phosphatidylethanolamines, phosphatidylglycerols and other examples of natural and synthetic lung surfactants; lauric acid and its salts, for example, sodium lauryl sulphate, magnesium lauryl sulphate; triglycerides such as Dynsan 118 and Cutina HR; and sugar esters in general. Alternatively, the coating material may comprise or consist of cholesterol. Other useful materials are film-forming agents, fatty acids and their derivatives, as well as lipids and lipid-like materials.

Other possible coating materials include sodium benzoate, hydrogenated oils which are solid at room temperature, talc, titanium dioxide, aluminium dioxide, silicon dioxide and starch.

In some embodiments, a plurality of different coating materials can be used, either as a mixture or as separate coating layers.

The coating material may be applied to the particles comprising the active agent in a variety of different ways. Preferably, the coating method adopted will result in a complete or substantially complete coating of the surface of the active particle, so as to completely cover and hide the active agent. The coating may be applied in a variety of different ways which would all be well known to the person skilled in the art of preparing active particles for inclusion in formulations for administration by pulmonary inhalation. Such coating methods include, for example, co-milling the materials, using compressive type techniques like Mechano-Fusion (which is discussed in detail later in this description), co-spray drying, and the like.

In a preferred embodiment, the active agent is clomipramine and the coating consists of or comprises magnesium stearate. The production of formulations comprising clomipramine and magnesium stearate using co-jet milling or Mechano-Fusion is described in the Examples. Inhalation of clomipramine formulations such as those described in the Examples is not believed to induce an involuntary cough because the clomipramine is prevented from contacting the chemosensors responsible for drug-induced coughs by the magnesium stearate coat.

The formulations of the invention may be administered with one or mote cough suppressing agents. The cough suppressing agent may be administered at the same time as the cough-inducing pharmaceutically active agent. Alternatively or additionally, the cough suppressing agent may be administered before the formulation comprising the cough-inducing active agent.

Examples of suitable cough suppressing agents include menthol, eucalyptus oil and camphor, codeine, diphenhydramine, guaifenesin, levodropropizine, moguisteine, bronchodilators including β2 agonists such as terbutaline, capsazepine and lidocaine. In a preferred embodiment, the cough suppressing agent is administered by pulmonary inhalation. In another preferred embodiment, the cough suppressing agent is included in the formulation with the cough inducing pharmaceutically active agent.

The localized delivery of dry powder formulations to the lung is thought to have a localized dehydration effect upon the airways, which can cause or increase airway irritation and induce a cough reflex. Therefore, the co-administration of hydrating agents via steam nebulisers that are known for the treatment of asthma and dry cough may minimize the localized irritation and reduce the incidence of the involuntary cough. The hydrating agents are effectively acting as cough-suppressing agents in the context of the present invention.

The formulations of the invention may be prepared for administration via another route. It is the case with a number of cough-inducing active agents, including clomipramine, that the cough is not induced when the active agent is administered via an alternative route to pulmonary inhalation. Thus, for example, alternative routes may be selected and these are preferably those that allow rapid absorption of the active agent and rapid onset of the desired therapeutic effect. Particularly attractive alternative routes of administration include intranasal, transmucosal (preferably sublingual or buccal absorption), and subcutaneous administration. Transdermal patches are also an option for avoiding the involuntary cough associated with inhalation of the active agent.

In connection with clomipramine, formulations in the form of a slow release (transdermal) patch, nasal spray, suppository, eye drop and injection are envisaged for the treatment of various conditions. The compositions are proposed in solid, liquid or other appropriate dosage forms including tablets, capsules and solutions, using conventional pharmaceutically acceptable vehicles and techniques. Like formulations for inhalation, such presentations of clomipramine have the advantage that they avoid the unpleasant taste associated with the conventional oral forms of the drug.

In some embodiments of the present invention, the compositions further include one or more other pharmaceutically active agents, and preferably at least one agent which is useful in the treatment of respiratory disorders. Such agents include bronchodilators, for example β₂-agonists such as bambuterol, bitolterol, fenoterol, formoterol, levalbuterol, metaproterenol, pirbuterol, procaterol, salbutamol, salmeterol, terbutaline and the like; antimuscarinics such as ipratropium, ipratropium bromide, tiotropium, LAS-34273, glycopyrronium, glycopyrrolate and the like; xanthines such as aminophylline, theophylline and the like; and other respiratory agents such as ephedrine, epinephrine, isoetharine, isoproterenol, montelukast, pseudoephedrine, sibenadet and zafirlukast.

The compositions according to the present invention may also include steroids, such as, for example, alcometasone, beclomethasone, beclomethasone dipropionate, betamethasone, budesonide, ciclesonide, clobetasol, deflazacort, diflucortolone, desoxymethasone, dexamethasone, fludrocortisone, flunisolide, fluocinolone, fluometholone, fluticasone, fluticasone proprionate, hydrocortisone, mometasone, methylprednisolone, nandrolone decanoate, neomycin sulphate, prednisolone, rimexolone, triamcinolone and triamcinolone acetonide.

Other types of active agents that may be included in the compositions of the present invention include: mucolytics such as N-acetylcysteine, amiloride, dextrans, heparin, desulphated heparin, low molecular weight heparin and recombinant human DNase; matrix metalloproteinase inhibitors (MMPIs); leukotriene receptor antagonists; 5-lipooxygenase inhibitors; antibiotics; antineoplastics; peptides; vaccines; antitussives; nicotine; PDE3 inhibitors; PDE4 inhibitors; mixed PDE3/4 inhibitors; elastase inhibitors; and mast cell stabilizers such as sodium cromoglycate and nedocromil.

Details of the therapy according to the present invention will depend on various factors, such as the age, sex or condition of the patient, and the existence or otherwise of one or more concomitant therapies. The nature and severity of the condition will also have to be taken into account.

According to one embodiment, the compositions according to the present invention are dry powders for pulmonary administration by inhalation. Preferably, such dry powder compositions are dispensed using a dry powder inhaler (DPI).

The compositions according to the present invention may be administered using active or passive DPIs. It is important to note that a dry powder formulation should be tailored to the specific type of device used to dispense it, in order to provide efficient and consistent delivery of the active agent to the target sites in the respiratory tract. This is especially important where accurate and reproducible active particle deposition is to be relied upon to help reduce or avoid the involuntary cough being induced.

Preferably, for delivery to the lower respiratory tract or deep lung, the mass median aerodynamic diameter (MMAD) of the active particles in a dry powder composition is not more than 5 μm, and preferably not more than 2 μm, more preferably not more than 1 μm, and may be less than 0.75 μm, less than 0.5 μm or less than 0.1 μm. Especially for deep lung or systemic delivery, the active particles may have a size of 0.1 to 3 μm or 0.1 to 2 μm.

Ideally, at least 90% by weight of the active particles in a dry powder formulation should have an aerodynamic diameter of not more than 5 μm, preferably not more than 2 μm, more preferably not more than 1 μm, not more than 0.75 μm, not more than 0.5 μm, or even not more than 0.1 μm.

Fine particles, that is, those with an MMAD of less than 10 μm and smaller, tend to be increasingly thermodynamically unstable as their surface area to volume ratio increases, which provides an increasing surface free energy with this decreasing particle size, and consequently increases the tendency of particles to agglomerate and the strength of the agglomerate. In the inhaler, agglomeration of fine particles and adherence of such particles to the walls of the inhaler are problems that result in the fine particles leaving the inhaler as large, stable agglomerates, or being unable to leave the inhaler and remaining adhered to the interior of the inhaler, or even clogging or blocking the inhaler.

The uncertainty as to the extent of formation of stable agglomerates of the particles between each actuation of the inhaler, and also between different inhalers and different batches of particles, leads to poor dose reproducibility. Furthermore, the formation of agglomerates means that the MMAD of the active particles can be vastly increased, with agglomerates of the active particles not reaching the required part of the lung.

In an attempt to improve this situation and to provide a consistent fine particle fraction (FPF) and fine particle dose (FPD), dry powder formulations often include additive material. The additive material is intended to control the cohesion between particles in the dry powder formulation. It is thought that the additive material interferes with the weak bonding forces between the small particles, helping to keep the particles separated and reducing the adhesion of such particles to one another, to other particles in the formulation if present and to the internal surfaces of the inhaler device. Where agglomerates of particles are formed, the addition of particles of additive material decreases the stability of those agglomerates so that they are more likely to break up in the turbulent air stream created on actuation of the inhaler device, whereupon the particles are expelled from the device and inhaled. As the agglomerates break up, the active particles return to the form of small individual particles which are capable of reaching the lower lung.

However, the optimum stability of agglomerates to provide efficient drug delivery will depend upon the nature of the turbulence created by the particular device used to deliver the powder. Agglomerates will need to be stable enough for the powder to exhibit good flow characteristics during processing and loading into the device, whilst being unstable enough to release the active particles of respirable size upon actuation.

Preferably, the additive material is an anti-adherent material and it will tend to reduce the cohesion between particles and will also prevent fine particles becoming attached to the inner surfaces of the inhaler device. Advantageously, the additive material is an anti-friction agent or glidant and will give better flow of the pharmaceutical composition in the inhaler. The additive materials used in this way may not necessarily be usually referred to as anti-adherents or anti-friction agents, but they will have the effect of decreasing the cohesion between the particles or improving the flow of the powder. The additive materials are often referred to as force control agents (FCAs) and they usually lead to better dose reproducibility and higher fine particle fractions. Therefore, a FCA, as used herein, is an agent whose presence on the surface of a particle can modify the adhesive and cohesive surface forces experienced by that particle, in the presence of other particles. In general, its function is to reduce both the adhesive and cohesive forces. These additives and FCAs are the same as those discussed above in connection with the proposed protective coatings surrounding and shielding the particles comprising the cough-inducing active agent. The discussion of FCAs below relates to these agents being included in the formulation of the invention in order to improve the powder properties and targeted lung deposition.

Known FCAs usually consist of physiologically acceptable material, although the additive material may not always reach the lung. Preferred materials for use in dry powder compositions include amino acids, peptides and polypeptides having a molecular weight of between 0.25 and 1000 kDa and derivatives thereof.

It is particularly advantageous for the FCA to comprise an amino acid. The FCA may comprise or consist of one or more of any of the following amino acids: leucine, isoleucine, lysine, valine, methionine and phenylalanine. The FCA may be a salt or a derivative of an amino acid, for example aspartame or acesulfame K. Preferably, the FCA consists substantially of an amino acid, more preferably of leucine, advantageously L-leucine. The D- and DL-forms may also be used. The FCA may comprise Aerocine™, amino acid particles as disclosed in the earlier patent application published as WO 00/33811.

The FCA may comprise or consist of dipolar ions, which may be zwitterions. It is also advantageous for the FCA to comprise or consist of a spreading agent, to assist with the dispersal of the composition in the lungs. Suitable spreading agents include surfactants such as known lung surfactants (e.g. ALEC®) which comprise phospholipids, for example, mixtures of dipalmitoyl phosphatidylcholine (DPPC) and phosphatidylglycerol (PG). Other suitable surfactants include, for example, dipalmitoyl phosphatidylethanolamine (DPPE) and dipalmitoyl phosphatidylinositol (DPPI).

Dry powder compositions often include carrier particles mixed with fine particles of active material. In such compositions, rather than sticking to one another, the fine active particles tend to adhere to the surfaces of the carrier particles whilst in the inhaler device, but are supposed to release and become dispersed upon actuation of the dispensing device and inhalation into the respiratory tract, to give a fine suspension. Such release may be improved by the inclusion of an FCA. The inclusion of coarse carrier particles is also very attractive where very small doses of active agent are dispensed. It is very difficult to accurately and reproducibly dispense very small quantities of powder and small variations in the amount of powder dispensed will mean large variations in the dose of active agent where the powder comprises mainly active particles. Therefore, the addition of a diluent, in the form of large excipient particles will make dosing more reproducible and accurate.

Carrier particles may comprise or consist of any acceptable excipient material or combination of materials and preferably the material(s) is (are) inert and physiologically acceptable. For example, the carrier particles may be composed of one or more materials selected from sugar alcohols, polyols and crystalline sugars. Other suitable carriers include inorganic salts such as sodium chloride and calcium carbonate, organic salts such as sodium lactate and other organic compounds such as polysaccharides and oligosaccharides. Advantageously the carrier particles are of a polyol. In particular the carrier particles may be particles of crystalline sugar, for example mannitol, dextrose or lactose. Preferably, the carrier particles are of lactose.

According to some embodiments of the present invention, the dry powder compositions include carrier particles that are relatively large, compared to the particles of active material. This means that substantially all (by weight) of the carrier particles have a diameter which lies between 20 μm and 1000 μm, or between 50 μm and 1000 μm. Preferably, the diameter of substantially all (by weight) of the carrier particles is less than 355 μm and lies between 20 μm and 250 μm. In one embodiment, the carrier particles have a MMAD of at least 90 μm.

Preferably, at least 90% by weight of the carrier particles have a diameter between from 60 μm to 180 μm. The relatively large diameter of the carrier particles improves the opportunity for other, smaller particles to become attached to the surfaces of the carrier particles and to provide good flow and entrainment characteristics and improved release of the active particles in the airways to increase deposition of the active particles in the lower lung.

Powder flow problems associated with compositions comprising larger amounts of fine material, such as up to from 5 to 20% by total weight of the formulation. This problem may be overcome by the use of large fissured lactose carrier particles, as discussed in earlier patent applications published as WO 01/78694, WO 01/78695 and WO 01/78696.

In other embodiments, the excipient or carrier particles included in the dry powder compositions are relatively small, having a median diameter of about 3 to about 40 μm, preferably about 5 to about 30 μm, more preferably about 5 to about 20 μm, and most preferably about 5 to about 15 μm. Such fine carrier particles, if untreated with an additive, are unable to provide suitable flow properties when incorporated in a powder composition comprising fine or ultra-fine active particles. Indeed, previously, particles in these size ranges would not have been regarded as suitable for use as carrier particles, and instead would only have been added in small quantities as a fine component in combination with coarse carrier particles, in order to increase the aerosolisation properties of compositions containing a drug and a larger carrier, typically with median diameter 40 μm to 100 μm or greater. However, the quantity of such a fine excipient may be increased and such fine excipient particles may act as carrier particles if these particles are treated with an additive or FCA, even in the absence of coarse carrier particles. Such treatment can bring about substantial changes in the powder characteristics of the fine excipient particles and the powders they are included in. Powder density is increased, even doubled, for example from 0.3 g/cc to over 0.5 g/cc. Other powder characteristics are changed, for example, the angle of repose is reduced and contact angle increased.

Treated fine carrier particles having a median diameter of 3 to 40 μm are advantageous as their relatively small size means that they have a reduced tendency to segregate from the drug component, even when they have been treated with an additive to reduce cohesion. This is because the size differential between the carrier and drug is relatively small compared to that in conventional compositions which include fine or ultra-fine active particles and much larger carrier particles. The surface area to volume ratio presented by the fine carrier particles is correspondingly greater than that of conventional large carrier particles. This higher surface area, allows the carrier to be successfully associated with higher levels of drug than for conventional larger carrier particles. This makes the use of treated fine carrier particles particularly attractive in powder compositions to be dispensed by passive devices.

The metered dose (MD) of a dry powder composition is the total mass of active agent present in the metered form presented by the inhaler device in question. For example, the MD might be the mass of active agent present in a capsule for a Cyclohaler™, or in a foil blister in a Gyrohaler™ device.

The emitted dose (ED) is the total mass of the active agent emitted from the device following actuation. It does not include the material left on the internal or external surfaces of the device, or in the metering system including, for example, the capsule or blister. The ED is measured by collecting the total emitted mass from the device in an apparatus frequently identified as a dose uniformity sampling apparatus (DUSA), and recovering this by a validated quantitative wet chemical assay (a gravimetric method is possible, but this is less precise).

The FPD is the total mass of active agent which is emitted from the device following actuation which is present in an aerodynamic particle size smaller than a defined limit. This limit is generally taken to be 5 μm if not expressly stated to be an alternative limit, such as 3 μm, 2 μm or 1 μm, etc. The FPD is measured using an impactor or impinger, such as a twin stage impinger (TSI), multi-stage impinger (MSI), Andersen Cascade Impactor (ACI) or a Next Generation Impactor (NGI).

Each impactor or impinger has a pre-determined aerodynamic particle size collection cut points for each stage. The FPD value is obtained by interpretation of the stage-by-stage active agent recovery quantified by a validated quantitative wet chemical assay (a gravimetric method is possible, but this is less precise) where either a simple stage cut is used to determine FPD or a more complex mathematical interpolation of the stage-by-stage deposition is used.

The FPF is normally defined as the FPD divided by the ED and expressed as a percentage. Herein, the FPF of ED is referred to as FPF(ED) and is calculated as FPF(ED)=(FPD/ED)×100%.

The FPF may also be defined as the FPD divided by the MD and expressed as a percentage. Herein, the FPF of MD is referred to as FPF(MD), and is calculated as FPF(MD)=(FPD/MD)×100%.

In one embodiment of the invention, the composition is a dry powder which has a fine particle fraction (<5 μm) of at least 50%, preferably at least 60%, at least 70% or at least 80%.

Preferably, these FPFs are achieved when the composition is dispensed using an active DPI, although such good FPFs may also be achieved using passive DPIs, especially where the device is one as described in the earlier patent application published as WO 2005/037353 and/or the dry powder composition has been formulated specifically for administration by a passive device.

In one embodiment of the invention, the DPI is an active device, in which a source of compressed gas or alternative energy source is used. Examples of suitable active devices include Aspirair™(Vectura) and the active inhaler device produced by Nektar Therapeutics (as disclosed in U.S. Pat. No. 6,257,233), and the ultrasonic Microdose™ or Oriel™devices.

In an alternative embodiment, the DPI is a passive device, in which the patient's breath is the only source of gas which provides a motive force in the device.

Examples of “passive” dry powder inhaler devices include the Rotahaler™ and Diskhaler™ (GlaxoSmithKline) and the Turbohaler™ (Astra-Draco) and Novolizer™ (Viatris GmbH) and GyroHaler™ (Vectura).

The dry powder formulations may be pre-metered and kept in capsules or foil blisters which offer chemical and physical protection whilst not being detrimental to the overall performance. Alternatively, the dry powder formulations may be held in a reservoir-based device and metered on actuation. Examples of “reservoir-based” inhaler devices include the Clickhaler™ (Innovata) and Duohaler™ (Innovata), and the Turbohaler™ (Astra-Draco). Actuation of such reservoir-based inhaler devices can comprise passive actuation, wherein the patient's breath is the only source of energy which generates a motive force in the device.

The particles of active agent included in the compositions of the present invention, may be formulated with additional excipients to aid delivery or to control release of the active agent upon deposition within the lung. In such embodiments, the active agent may be embedded in or dispersed throughout particles of an excipient material which may be, for example, a polysaccharide matrix. Alternatively, the excipient may form a coating, partially or completely surrounding the particles of active material. Upon delivery of these particles to the lung, the excipient material acts as a temporary barrier to the release of the active agent, providing a delayed or sustained release of the active agent. Suitable excipient materials for use in delaying or controlling the release of the active material will be well known to the skilled person and will include, for example, pharmaceutically acceptable soluble or insoluble materials such as polysaccharides, for example xanthan gum. A dry powder composition may comprise the active agent in the form of particles which provide immediate release, as well as particles exhibiting delayed or sustained release, to provide any desired release profile.

Compositions according to the invention may be produced using conventional formulation techniques.

Spray drying is a well-known and widely used technique for producing particles of active material of inhalable size. Conventional spray drying techniques may be improved so as to produce active particles with enhanced chemical and physical properties so that they perform better when dispensed from a DPI than particles formed using conventional spray drying techniques. Such improvements are described in detail in the earlier patent application published as WO 2005/025535.

In particular, it is disclosed that co-spray drying an active agent with an FCA under specific conditions can result in particles with excellent properties which perform extremely well when administered by a DPI for inhalation into the lung.

It has been found that manipulating or adjusting the spray drying process can result in the FCA being largely present on the surface of the particles. That is, the FCA is concentrated at the surface of the particles, rather than being homogeneously distributed throughout the particles. This clearly means that the FCA will be able to reduce the tendency of the particles to agglomerate. This will assist the formation of unstable agglomerates that are easily and consistently broken up upon actuation of a DPI.

It has been found that it may be advantageous to control the formation of the droplets in the spray drying process, so that droplets of a given size and of a narrow size distribution are formed. Furthermore, controlling the formation of the droplets can allow control of the air flow around the droplets which, in turn, can be used to control the drying of the droplets and, in particular, the rate of drying. Controlling the formation of the droplets may be achieved by using alternatives to the conventional 2-fluid nozzles, especially avoiding the use of high velocity air flows. In particular, it is preferred to use a spray drier comprising a means for producing droplets moving at a controlled velocity and of a predetermined droplet size. The velocity of the droplets is preferably controlled relative to the body of gas into which they are sprayed. This can be achieved by controlling the droplets' initial velocity and/or the velocity of the body of gas into which they are sprayed, for example by using an ultrasonic nebuliser (USN) to produce the droplets.

Alternative nozzles such as electrospray nozzles or vibrating orifice nozzles may be used.

Spray drying may be used to produce microparticles comprising or consisting of the active agent (being clomipramine alone or in combination with any other therapeutically active agent(s)). In some embodiments, the spray drying process may be adapted to produce spray-dried particles that include the active agent dispersed or suspended within a material that provides the controlled release properties.

The process of milling, for example, jet milling, may also be used to formulate the dry powder compositions according to the present invention. The manufacture of fine particles by milling can be achieved using conventional techniques. In the conventional use of the word, “milling” means the use of any mechanical process which applies sufficient force to the particles of active material that it is capable of breaking coarse particles (for example, particles with a MMAD greater than 100 μm) down to fine particles (for example, having a MMAD not more than 50 μm). In the present invention, the term “milling” also refers to deagglomeration of particles in a formulation, with or without particle size reduction. The particles being milled may be large or fine prior to the milling step. A wide range of milling devices and conditions are suitable for use in the production of the compositions of the inventions. The selection of appropriate milling conditions, for example, intensity of milling and duration, to provide the required degree of force will be within the ability of the skilled person. Ball milling is a preferred method. Alternatively, a high pressure homogeniser may be used in which a fluid containing the particles is forced through a valve at high pressure producing conditions of high sheer and turbulence. Sheer forces on the particles, impacts between the particles and machine surfaces or other particles, and cavitation due to acceleration of the fluid may all contribute to the fracture of the particles. Suitable homogenisers include the EmulsiFlex high pressure homogeniser, the Niro Soavi high pressure homogeniser and the Microfluidics Microfluidiser. The milling process can be used to provide the microparticles with mass median aerodynamic diameters as specified above.

Milling the active agent with a FCA and/or with a material which can delay or control the release of the active agent is preferred. Co-milling or co-micronising particles of active agent and particles of FCA or excipient will result in the FCA or excipient becoming deformed and being smeared over or fused to the surfaces of fine active particles. These resultant composite active particles comprising an FCA have been found to be less cohesive after the milling treatment. If a significant reduction in particle size is also required, co-jet milling is preferred, as disclosed in the earlier patent application published as WO 2005/025536. The co-jet milling process can result in composite active particles with low micron or sub-micron diameter, and these particles exhibit particularly good FPF and FPD, even when dispensed using a passive DPI.

The milling processes apply a high enough degree of force to break up tightly bound agglomerates of fine or ultra-fine particles, such that effective mixing and effective application of the additive material to the surfaces of those particles is achieved.

In one embodiment, if required, the microparticles produced by the milling step can then be formulated with an additional excipient. This may be achieved by a spray drying process, e.g. co-spray drying. In this embodiment, the particles are suspended in a solvent and co-spray dried with a solution or suspension of the additional excipient. Preferred additional excipients include polysaccharides. Additional pharmaceutical effective excipients may also be used.

The prior art mentions two types of processes in the context of co-milling or co-micronising active and additive particles.

First, there is the compressive type process, such as Mechano-Fusion and Cyclomix methods, as well as related methods such as those involving the use of a Hybridiser or the Nobilta. As the name suggests, Mechano-Fusion is a dry coating process designed to mechanically fuse a first material onto a second material. It should be noted that the use of the terms “Mechano-Fusion” and “Mechanofused” are supposed to be interpreted as a reference to a particular type of milling process, but not a milling process performed in a particular apparatus. The first material is generally smaller and/or softer than the second. The Mechano-Fusion and Cyclomix working principles are distinct from alternative milling techniques in having a particular interaction between an inner element and a vessel wall, and are based on providing energy by a controlled and substantial compressive force.

The fine active particles and the additive particles are fed into the Mechano-Fusion driven vessel (such as a Mechano-Fusion system (Hosokawa Micron Ltd)), where they are subject to a centrifugal force and are pressed against the vessel inner wall. The powder is compressed between the fixed clearance of the drum wall and a curved inner element with high relative speed between drum and element. The inner wall and the curved element together form a gap or nip in which the particles are pressed together. As a result, the particles experience very high shear forces and very strong compressive stresses as they are trapped between the inner drum wall and the inner element (which has a greater curvature than the inner drum wall). The particles are pressed against each other with enough energy to locally heat and soften, break, distort, flatten and wrap the additive particles around the core particle to form a coating. The energy is generally sufficient to break up agglomerates and some degree of size reduction of both components may occur.

These Mechano-Fusion and Cyclomix processes apply a high enough degree of force to separate the individual particles of active-material and to break up tightly bound agglomerates of the active particles such that effective mixing and effective application of the additive material to the surfaces of those particles is achieved. An especially desirable aspect of the described co-milling processes is that the additive material becomes deformed in the milling and may be smeared over or fused to the surfaces of the active particles.

However, in practice, this compression process produces little or no milling (i.e. size reduction) of the drug particles, especially where they are already in a micronised form (i.e. <10 μm), the only physical change which may be observed is a plastic deformation of the particles to a rounder shape.

Secondly, there are the impact milling processes involved in ball milling and the use of a homogeniser.

Ball milling is a suitable milling method for use in the prior art co-milling processes. Centrifugal and planetary ball milling are especially preferred methods. Alternatively, a high pressure homogeniser may be used in which a fluid containing the particles is forced through a valve at high pressure producing conditions of high shear and turbulence. Such homogenisers may be more suitable than ball mills for use in large scale preparations of the composite active particles.

Suitable homogenisers include EmulsiFlex high pressure homogenisers which are capable of pressures up to 4000 bar, Niro Soavi high pressure homogenisers (capable of pressures up to 2000 bar), and Microfluidics Microfluidisers (maximum pressure 2750 bar). The milling step may, alternatively, involve a high energy media mill or an agitator bead mill, for example, the Netzsch high energy media mill, or the DYNO-mill (Willy A. Bachofen A G, Switzerland).

These processes create high-energy impacts between media and particles or between particles. In practice, while these processes are good at making very small particles, it has been found that neither the ball mill nor the homogeniser was effective in producing dispersion improvements in resultant drug powders in the way observed for the compressive process. It is believed that the second impact processes are not as effective in producing a coating of additive material on each particle.

Conventional methods comprising co-milling active material with additive materials (as described in WO 02/43701) result in composite active particles which are fine particles of active material with an amount of the additive material on their surfaces. The additive material is preferably in the form of a coating on the surfaces of the particles of active material. The coating may be a discontinuous coating. The additive material may be in the form of particles adhering to the surfaces of the particles of active material.

At least some of the composite active particles may be in the form of agglomerates. However, when the composite active particles are included in a pharmaceutical composition, the additive material promotes the dispersal of the composite active particles on administration of that composition to a patient, via actuation of an inhaler.

Jet mills are capable of reducing solids to particle sizes in the low-micron to submicron range. The grinding energy is created by gas streams from horizontal grinding air nozzles. Particles in the fluidised bed created by the gas streams are accelerated towards the centre of the mill, colliding with slower moving particles. The gas streams and the particles carried in them create a violent turbulence and as the particles collide with one another they are pulverised.

In the past, jet-milling has not been considered attractive for co-milling active and additive particles, processes like Mechano-Fusion and Cyclomixing being clearly preferred. The collisions between the particles in a jet mill are somewhat uncontrolled and those skilled in the art, therefore, considered it unlikely for this technique to be able to provide the desired deposition of a coating of additive material on the surface of the active particles. Moreover, it was believed that, unlike the situation with Mechano-Fusion and Cyclomixing, segregation of the powder constituents occurred in jet mills, such that the finer particles, that were believed to be the most effective, could escape from the process. In contrast, it could be clearly envisaged how techniques such as Mechano-Fusion would result in the desired coating.

It should also be noted that it was also previously believed that the compressive or impact milling processes must be carried out in a closed system, in order to prevent segregation of the different particles. This has also been found to be untrue and the co-jet milling processes according to the present invention do not need to be carried out in a closed system. Even in an open system, the co-jet milling has surprisingly been found not to result in the loss of the small particles, even when using leucine as the additive material.

It has now unexpectedly been discovered that composite particles of active and additive material can be produced by co-jet milling these materials. The resultant particles have excellent characteristics which lead to greatly improved performance when the particles are dispensed from a DPI for administration by inhalation. In particular, co-jet milling active and additive particles can lead to further significant particle size reduction.

The effectiveness of the promotion of dispersal of active particles has been found to be enhanced by using the co-jet milling methods in comparison to compositions which are made by simple blending of similarly sized particles of active material with additive material. The phrase “simple blending” means blending or mixing using conventional tumble blenders or high shear mixing and basically the use of traditional mixing apparatus which would be available to the skilled person in a standard laboratory.

In another embodiment, the particles produced using the co-jet milling processes discussed above subsequently undergo Mechano-Fusion. This final Mechano-Fusion step is thought to “polish” the composite active particles, further rubbing the additive material into the particles. This allows one to enjoy the beneficial properties afforded to particles by Mechano-Fusion, in combination with the very small particles sizes made possible by the co-jet milling.

The dry powder compositions of the present invention may benefit from including relatively dense particles of active agent (clomipramine and any other pharmaceutically active material included). Thus, powders according to some embodiments of the present invention may preferably have a tapped density of at least 0.1 g/cc, at least 0.2 g/cc, at least 0.3 g/cc, at least 0.4 g/cc, or at least 0.5 g/cc. The inclusion of such relatively dense particles of active material in dry powder compositions unexpectedly leads to good FPFs and FPDs and these dense particles may help reduce the volume of powder that must be administered to the lung.

In a yet further embodiment, the composition is a solution or suspension and is administered using a pressurised metered dose inhaler (pMDI), a nebuliser or a soft mist inhaler. Examples of suitable devices include pMDIs such as Modulite® (Chiesi), SkyeFine™ and SkyeDry™ (SkyePharma). Nebulisers such as Porta-Neb®, Inquaneb™ (Pari) and Aquilon™, and soft mist inhalers such as eFlow™ (Pari), Aerodose™ (Aerogen), Respimat® Inhaler (Boehringer Ingelheim GmbH), AERx® Inhaler (Aradigm) and Mystic™ (Ventaira Pharmaceuticals, Inc.).

Where the compositions are to be dispensed using a pMDI, the compositions comprising hydroxychloroquine preferably further comprises a propellant. In embodiments of the present invention, the propellant is CFC-12 or an ozone-friendly, non-CFC propellant, such as 1,1,1,2-tetrafluoroethane (HFC 134a), 1,1,1,2,3,3,3-heptafluoropropane (HFC-227), HCFC-22 (difluororchloromethane), HFA-152 (difluoroethane and isobutene) or combinations thereof. Such formulations may require the inclusion of a polar surfactant such as polyethylene glycol, diethylene glycol monoethyl ether, polyoxyethylene sorbitan monolaurate, polyoxyethylene sorbitan monooleate, propoxylated polyethylene glycol, and polyoxyethylene lauryl ether for suspending, solubilizing, wetting and emulsifying the active agent and/or other components, and for lubricating the valve components of the MDI.

Where the composition is to be dispensed using a nebuliser or soft mist inhaler, the composition is in the form of a solution or suspension. Thus, in some embodiments, these compositions comprise a solvent and/or water.

In one embodiment, an ultrasonic nebuliser (USN) is used to form the droplets in the spray mist. USNs use an ultrasonic transducer which is submerged in a liquid. The ultrasonic transducer (a piezoelectric crystal) vibrates at ultrasonic frequencies to produce the short wavelengths required for liquid atomisation. In one common form of USN, the base of the crystal is held such that the vibrations are transmitted from its surface to the nebuliser liquid, either directly or via a coupling liquid, which is usually water. When the ultrasonic vibrations are sufficiently intense, a fountain of liquid is formed at the surface of the liquid in the nebuliser chamber. Droplets are emitted from the apex and a “fog” emitted.

The invention will now be further described with reference to the following Examples.

### EXAMPLES

The following Examples describe suitable methods for producing the formulations of the invention. In the following Examples, the formulations are produced from a commercially available clomipramine hydrochloride powder, using either the Hosokawa AS50 or AS100 jet mill. Clomipramine hydrochloride may be milled alone or with a coating agent such as magnesium stearate.

### Example 1

### Clomipramine Hydrochloride Milled Alone

Pure clomipramine hydrochloride was passed through a Hosokawa AS50 jet mill three times, each time with an injector air pressure of 6 bar, grinding air pressure of 4 bar and powder feed rate of approximately 3 g/min. Malvern (dry powder) particle size measurement of the resultant powder gave a D₅₀ of 0.973 μm.

### Example 2

### Clomipramine Hydrochloride Jet-Milled with Magnesium Stearate

Unmicronised clomipramine hydrochloride and magnesium stearate were mixed in a respective ratio of 98:2 (formulation A) or 95:5 (formulation B) using a WAB Turbula mixer for approximately 10 minutes at 32 rpm. Each mixture was then co-milled in a Hosokawa AS50 Spiral Jet Mill using a feed rate of approximately 3 g/min, a Venturi pressure of 6 bar and a grinding air pressure of 4 bar. Each co-milled formulation was recovered from the filter bag and collection vessel, and sieved through a 315 μm sieve screen. Malvern (dry powder) particle size measurement of formulation A gave a D₅₀ of 0.882 μm and that of formulation B gave a D₅₀ of 1.231 μm.

This method may also be used to prepare a formulation from unmicronised clomipramine hydrochloride and magnesium stearate in a respective ratio of 90:10 (formulation C).

### Example 3

### Clomipramine Hydrochloride Jet-Milled with Magnesium Stearate and Combined with Lactose Carrier

Unmicronised clomipramine hydrochloride was mixed with magnesium stearate in a respective ratio of 98:2 (formulation A) or 95:5 (formulation B) using a WAB Turbula mixer for approximately 10 minutes at 32 rpm. Each mixture was co-milled in a Hosokawa AS50 Spiral Jet Mill using a feed rate of approximately 3 g/min, a Venturi pressure of 6 bar, and a grinding air pressure of 4 bar. Each co-milled formulation was recovered from the filter bag and collection vessel, and sieved through a 315 μm sieve screen. Malvern (dry powder) particle size measurement of formulation A gave a D₅₀ of 0.882 μm and that of formulation B gave a D₅₀ of 1.231 μm.

Each co-milled formulation is then mixed with Respitose (lactose carrier) in a respective ratio of 80:20 using a Diosna P1/6 Pharma Mixer as follows: half of the Respitose is placed in a one litre bowl, and the co-milled formulation is added thereto. The remaining Respitose is then added and the ingredients are mixed for one minute at 200 rpm, one minute at 250 rpm (Mixing impellor speed), and 10 minutes at 1000 rpm (Mixing impellor speed). At one-minute intervals, the sides of the mixing bowl are scraped down using a pallet knife to incorporate adhered material into the ingredients to be mixed. The mixture is sieved through a 160 μm sieve screen and returned to the mixing bowl. The final clomipramine hydrochloride and magnesium stearate concentrations in the mixture are 78.4% w/w and 1.6% w/w, respectively (formulation A) and 76% w/w and 4% w/w, respectively (formulation B).

This method may also be used to prepare a formulation from unmicronised clomipramine hydrochloride and magnesium stearate in a respective ratio of 90:10 (formulation C). The final clomipramine hydrochloride and magnesium stearate concentrations in the mixture with Respitose are 72% w/w and 8% w/w, respectively.

### Example 4

### Clomipramine Hydrochloride Jet-Milled with Magnesium Stearate and then Finally Processed Using Mechano-Chemical Bonding

Unmicronised clomipramine hydrochloride was mixed with magnesium stearate in a respective ratio of 98:2 (formulation A) or 95:5 (formulation B) using a WAB Turbula mixer for approximately 10 minutes at 32 rpm. The mixture was co-milled in a Hosokawa AS50 Spiral Jet Mill using nitrogen gas, a feed rate of approximately 3 g/min, a Venturi pressure of 6 bar, and a grinding pressure of 4 bar. Each co-milled formulation was recovered from the filter bag and collection vessel, and sieved through a 315 μm sieve screen. Malvern (dry powder) particle size measurement of formulation A gave a D₅₀ of 0.882 μm and that of formulation B gave a D₅₀ of 1.231 μm.

This method may also be used to prepare a formulation from unmicronised clomipramine hydrochloride and magnesium stearate in a respective ratio of 90:10 (formulation C).

Each co-milled sample is then added to the mechanofusion system (Hosokawa Micron ‘Mini Kit’ with a 3 mm rotor gap size) in sub-batches of 30-40 g with the system running at approximately 250 rpm. Each sub-batch is pre-mixed in the mechanofusion system for five minutes (mixing speed of approximately 1000 rpm), then, whilst remaining in the Hosokawa Micron ‘Mini Kit’, mechanofused for 10 minutes (mixing speed of approximately 4000 rpm). The resultant sub-batches are combined by mixing in a Turbula mixer for five minutes at 30 rpm to produce each final formulation.

1. A pharmaceutical composition for pulmonary inhalation comprising a cough-inducing pharmaceutically active agent, said agent being present in particles, the majority of which are of a size appropriate for deposition in the deep lung.

2. A composition as claimed in claim 1, wherein the cough-inducing pharmaceutically active agent is clomipramine or a pharmaceutically acceptable salt thereof.

3. A composition as claimed in claim 1, wherein the D₅₀ of the particles is less than about 2 μm.

4. A composition as claimed in claim 1, wherein the D₅₀ of the particles is less than about 1.5 μm.

5. A composition as claimed in claim 1, wherein the D₅₀ of the particles is less than about 1 μm.

6. A composition as claimed in claim 1, wherein the geometric standard deviation of the active particle size distribution is not more than about 2.

7. A composition as claimed in claim 1, wherein the geometric standard deviation of the active particle size distribution is not more than about 1.5.

8. A composition as claimed in claim 1, wherein the geometric standard deviation of the active particle size distribution is not more than about 1.2.

9. A pharmaceutical composition for pulmonary inhalation comprising a cough-inducing pharmaceutically active agent which is shielded by a coating.

10. A composition as claimed in claim 9, wherein the coating is a complete or a substantially complete coating.

11. A composition as claimed in claim 9, wherein the cough-inducing pharmaceutically active agent is clomipramine or a pharmaceutically acceptable salt thereof.

12. A composition as claimed in claim 9, wherein the coating comprises a metal stearate selected from the group consisting of zinc stearate, magnesium stearate, calcium stearate, sodium stearate and lithium stearate.

13. A composition as claimed in claim 9, wherein the coating comprises magnesium stearate.

14. A composition as claimed in claim 9, wherein the cough-inducing pharmaceutically active agent is present in particles, the majority of which are of a size appropriate for deposition in the deep lung.

15. A composition as claimed in claim 14, wherein the mass median aerodynamic diameter of the active particles is not more than about 5 μm.

16. A composition as claimed in claim 14, wherein the mass median aerodynamic diameter of the active particles is not more than about 2 μm.

17. A composition as claimed in claim 14, wherein the mass median aerodynamic diameter of the active particles is not more than about 1 μm.

18. A composition as claimed in claim 1, which is a dry powder composition.

19. A composition as claimed in claim 18, further comprising carrier particles.

20. A composition as claimed in claim 18, further comprising additive material.

21. A composition as claimed claim 18, produced using spray drying, milling, homogenisation, Cyclomixing or Mechano-Fusion.

22. A composition as claimed in claim 9, wherein the coating consists of a metal stearate selected from the group consisting of zinc stearate, magnesium stearate, calcium stearate, sodium stearate and lithium stearate.

23. A composition as claimed in claim 9, wherein the coating consists of magnesium stearate.

24. A composition as claimed in claim 9, which is a dry powder composition.

25. A composition as claimed in claim 24, further comprising carrier particles.

26. A composition as claimed in claim 24, further comprising additive material.

27. A composition as claimed in claim 24, produced using spray drying, milling, homogenisation, Cyclomixing or Mechano-Fusion.
